(19) United States
(12) Patent Application Publication
(10) Pub. No.: US 2008/0060647 A1
(43) Pub. Date: Mar. 13, 2008

(54) System and method for delivering a breathing gas

(75) Inventors: Robert W. Messenger, Lakewood, OH (US); David G. Felty, Parma, OH (US)

Correspondence Address: CALFEE HALTER & GRISWOLD, LLP, 800 SUPERIOR AVENUE, SUITE 1400, CLEVELAND, OH, 44114, US

(73) Assignee: Invacare Corporation

(21) Appl. No.: 11/519,532

(22) Filed: Sep. 12, 2006

(51) Int. Cl. A61M 16/00; A62B 7/00

(52) U.S. Cl. 128/204.23; 128/204.18

(57) ABSTRACT

Systems and methods delivering a breathing gas are provided. The methods include, for example, generating a first pressure level of breathing gas, increasing the pressure level of the breathing gas from the first pressure level to a second pressure level over a duration greater than one night's sleep, and maintaining the second pressure level over a duration of at least one night's sleep.

### BACKGROUND

Obstructive sleep apnea is a sleep-related breathing disorder caused by relaxation of the muscles of the upper airway to the point where the upper airway either fully or partially collapses or becomes obstructed. It is known that obstructive sleep apnea can be treated through the application of pressurized air or breathing gas to the nasal passages of a patient. The application of pressurized air forms a pneumatic splint in the upper airway of the patient thereby preventing the collapse or obstruction thereof. There are also other beneficial aspects to the application of a positive air pressure to the patient such as, for example, reducing cardiac preload and afterload on the heart.

### SUMMARY

A method of delivering a breathing gas is provided. The method includes, for example, generating a first pressure level of breathing gas, increasing the pressure level of the breathing gas from the first pressure level to at least a second pressure level over a duration greater than one night's sleep, and maintaining the second pressure level over a duration of at least one night's sleep.

### BRIEF DESCRIPTION OF THE DRAWINGS

FIG. 1 is an exemplary system diagram in accordance with one embodiment;

FIGS. 2A through 7 are pressure versus time graphs in accordance with various embodiments; and

FIGS. 8 through 10 are flowcharts illustrating embodiments of logic or instructions.

### DETAILED DESCRIPTION OF ILLUSTRATED EMBODIMENTS

The following includes definitions of exemplary terms used throughout the disclosure and specification. Both singular and plural forms of all terms fall within each meaning:

“Signal”, as used herein includes, but is not limited to, one or more electrical signals, analog or digital signals, optical or light (electro-magnetic) signals, one or more computer instructions, a bit or bit stream, or the like.

“Night's Sleep”, as used herein includes, but is not limited to, a period of time in which the natural periodic suspension of consciousness occurs during which the powers of the body are restored and is not limited to day or night or a single occurrence. For example, a typical “night's sleep” may include the period during which an individual retires for the evening to his or her bed. This time span may be characterized by periods of wakefulness, partial sleep, sleep, disruptive sleep and other various stages of sleep. Hence, a night's sleep may be characterized by states of consciousness and partial or complete suspension of consciousness. A “night's sleep” can also be characterized by calendar days, hours, or minutes. For example, one night's sleep can be characterized by one calendar day. Similarly, two night's sleep can be characterized by two calendar days.

“Logic”, synonymous with “circuit” as used herein, includes but is not limited to hardware, firmware, software and/or combinations of each to perform a function(s) or an action(s). For example, based on a desired application or needs, logic may include a software controlled microprocessor, discrete logic such as an application specific integrated circuit (ASIC), or other programmed logic device. Logic may also be fully embodied as software. “Software”, as used herein, includes but is not limited to one or more computer readable and/or executable instructions that cause a computer or other electronic device to perform functions, actions, and/or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software may be dependent on, for example, requirements of a desired application, the environment it runs on, and/or the desires of a designer/programmer or the like.

“Time period” includes, but is not limited to, an interval of time characterized by the occurrence of a certain condition, event, or phenomenon or the lack thereof. “Time period” may also include the measure of an interval, which can be any number represented by year(s), month(s), week(s), day(s), or minute(s), or combinations of the foregoing. Examples of a time period include 1 month, 1 to 3 months, 2 weeks, 14 days, 14 to 16 days, 6 hours, 6 to 8 hours, 122 minutes, 120 to 180 minutes, 1 month and 3 weeks, 5 nights, 5-7 nights, etc.

“Incrementally” includes, but is not limited to, changing by a series of events that include quantity, value or extent; by at least one of a series of regular consecutive additions, subtractions or modifications; or by an amount or degree.

“Ramp function” includes, but is not limited to, increasing or decreasing by a constant or non-constant bend, slope, or curve; or a change in level or direction by a constant or non-constant slope.

“Usage data” includes, but is not to limited to, any form of data including combinations of data indicating the usage of a machine, therapy, modality, or feature. For example, “usage data” can include compliance data indicating the time during which a particular therapy was used by a patient. The data can be in any form such as, for example, months, weeks, days, hours, minutes, seconds, or combinations of the foregoing.

“Threshold value” includes, but is not limited to, the point or range that must be exceeded to begin producing an effect or result or to elicit a response; or any value, level, or point that is used for setting a limit or condition.

“Sensor data” includes, but is not limited to, any form of data including digital and or analog that is produced by a device that measures, determines or tracks parameters or variables. “Sensor data” can include raw data or data that has been conditioned or modified subsequent to its generation. “Sensor data” can include, for example, flow rate data, temperature data, pressure data, physiological data (e.g., heart rate, blood pressure, EKG, breathing rate, arterial oxygen content, etc.), valve position data, motor speed or r.p.m. (rotations per minute) data, motor current signals or data, and/or motor voltage signals or data.

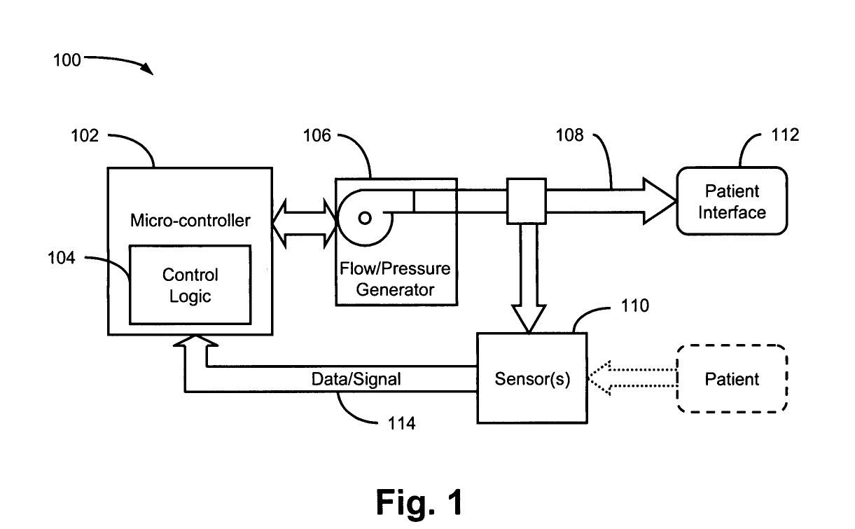

The systems and methods described herein are suited for assisting the respiration of spontaneously breathing patients, though they may also be applied to other respiratory regimens including, for example, acute and homecare ventilation. Referring now to FIG. 1, block diagram 100 illustrating one embodiment of a system is shown. The system has a controller 102 with control logic 104, a flow/pressure generator 106, and one or more sensor(s) 110. A flow path 108 provides a path for a flow of breathable gas from the flow/pressure generator 106 to a patient interface 112. Patent interface 112 can be any nasal mask, face mask, cannula, or similar device. Sensor(s) 112 can sense a parameter of the breathing gas such as the pressure in flow path 108, which is associated with and indicative of the pressure in the patient interface 112. The controller 102 is preferably processor-based and can have various input/output circuitry including analog-to-digital (A/D) inputs and digital-to-analog (D/A) outputs. The controller 102 sends data or signals indicative of flow/pressure control to flow/pressure generator 106 and the sensor(s) 110 send data or signals 114 back to the controller 102 to be read and analyzed. One embodiment of such as system is described in U.S. patent application Ser. Nos. 10/601,720 and 11/157,089, and U.S. Pat. No. 6,990,980, which are hereby fully incorporated by reference.

Flow/pressure generator 106 can be a variable speed blower alone or in combination with one or more valves such as, for example, a linear valve, solenoid valve, and/or a stepper motor controlled variable position valve. The flow/pressure generator 106 can also be a constant speed blower with a linear, solenoid and/or motor-driven stepper valve. The sensor element(s) 110 can include a flow sensor, flow rate sensor, temperature sensor, exhaled gas concentration sensor, infra-red light emitter/sensor, motor current sensor, or motor speed sensor alone or in combination with the pressure sensor. Sensor(s) 110 may also measure physiological parameters of a patient such as, for example, heart rate, blood pressure, EKG, breathing rate, arterial oxygen content, body temperature, brain wave activity, etc. The data generated from sensor(s) 110 is fed back to the controller 102 for processing.

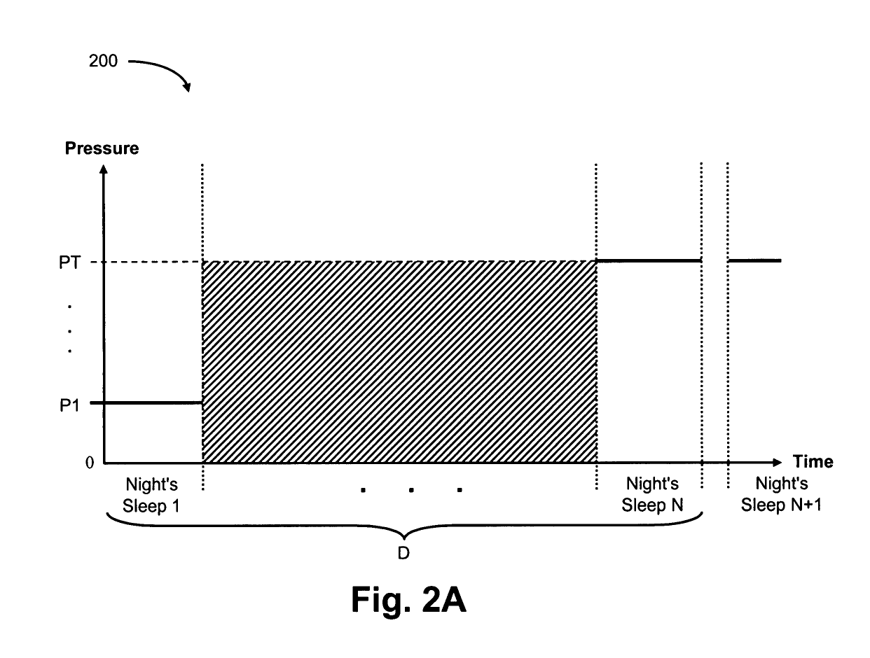

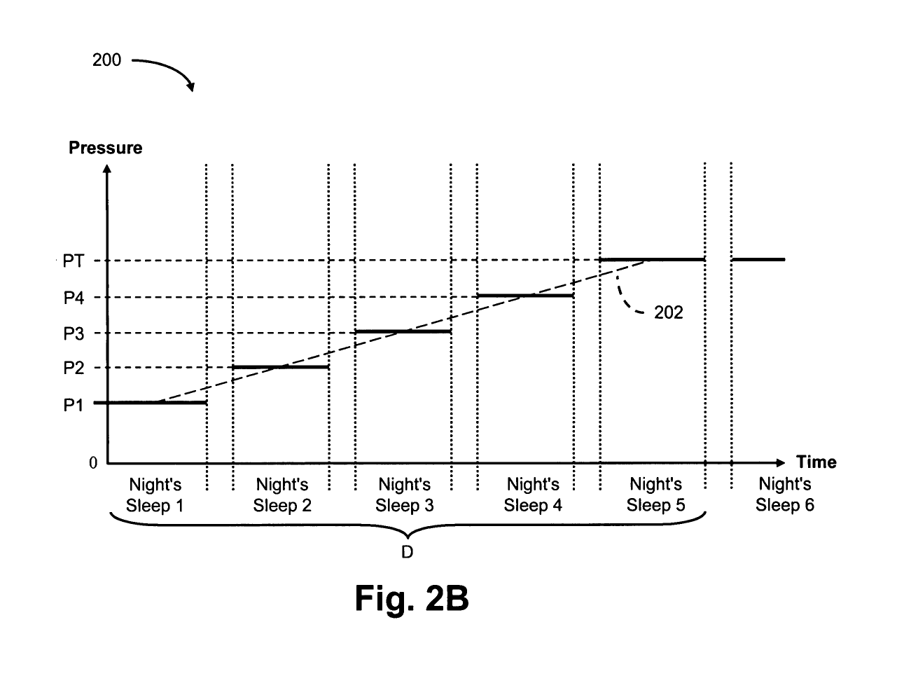

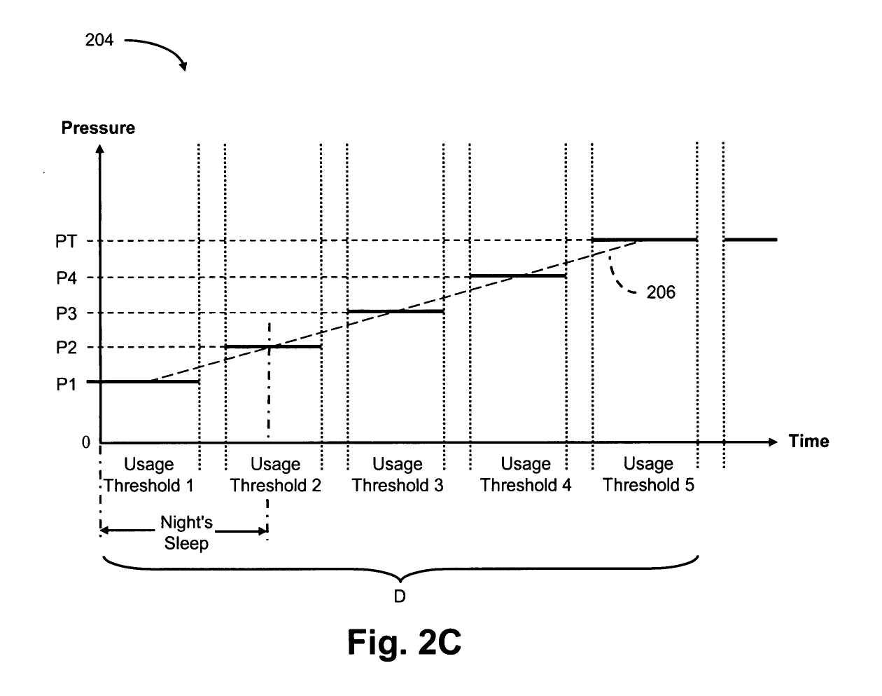

Control logic 104 includes instructions for generating one or more embodiments of the therapy modalities illustrated in FIGS. 2A through 7. FIG. 2A illustrates a general embodiment 200 of a pressure delivery modality. The pressure initially begins at pressure P1 and rises to therapy pressure PT over range a duration D that can be as short as two night's sleep, which is represented by removing the cross-hatched area in FIG. 2A, or as long as “N” night's sleep, which is represented by the cross-hatched area in FIG. 2A. The rise in pressure from pressure P1 to final therapy pressure PT can occur over a range of pressure changes as large as the changing from pressure P1 directly to final therapy pressure PT or through any number of intermediate pressure changes. Hence, the profile of the pressure changes from pressure P1 to final pressure therapy pressure PT can take an unlimited number of forms or representations.

FIG. 2B further illustrates embodiment 200 of a pressure delivery modality. In this embodiment, the system outputs a pressure that initially begins at pressure P1 and incrementally rises to therapy pressure PT. The rise from initial pressure P1 to final therapy pressure PT occurs over a duration D shown as five nights (hereinafter “Night's Sleep” shall be referred to as “Night”). The pressure output begins at pressure P1 on Night 1. At Night 2, the pressure is increased to P2. At Night 3, the pressure is increased to P3. At Night 4, the pressure is increased to P4. At Night 5, the pressure is increased to the final therapy pressure PT. During Night 6 and subsequent nights until the system is reset or changed, the output pressure remains at pressure PT. The increases in pressure from Night 1 to Night 5 are linear and incremental in this embodiment. This is represented by line segment or pressure profile 202 that has a constant positive slope. That is, the amount of pressure by which the output increases from night to night is the same over the duration D of five nights.

As will be described in the following embodiments, the change in initial pressure P1 to final pressure PT can occur over widely varying durations of time and according to constant and/or non-constant changes in pressure. While varying durations and profiles of pressure changes from initial pressure P1 to final therapy pressure PT will be described, the particular duration(s), initial pressure P1, a final therapy pressure PT, and profile of the pressure changes that are suitable for a particular patient are preferably set by a sleep therapist, respiratory therapist, sleep doctor, or other sleep professional. In one example, these settings may be set or adjusted through a interface such as a keypad. It is contemplated that the patient may set or adjust one or more of these parameters him or herself. It is also further contemplated that the system may set or adjust one or more of these parameters on its own in an automatic manner based on one or more feedback loops. It is also contemplated that settings or adjustments can occur through a remote or non-attached adjustment device that is in wireless communication with the system. It is also contemplated that settings or adjustments can occur through information that is downloaded to the memory of the system over a network, memory card, disk or similar device.

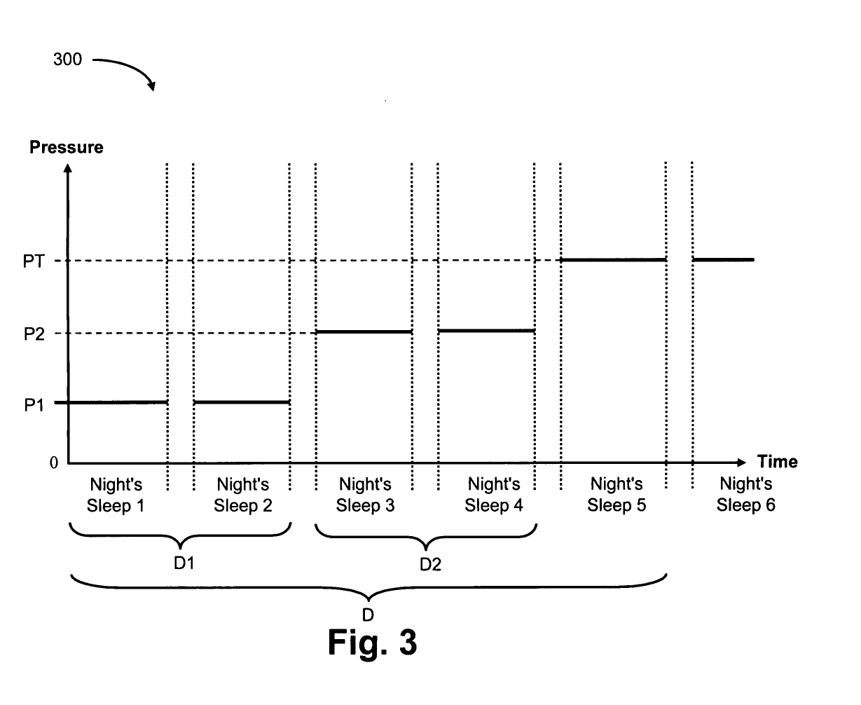

FIG. 3 illustrates an embodiment 300 similar to embodiment 200 except that the duration D includes sub-durations D1 and D2 that define the duration for each pressure increment. For example, pressure P1 is defined as lasting for duration D1 that is defined as nights 1 and 2. The next pressure increment, intermediate pressure P2, is defined as lasting for duration D2 that is defined as nights 3 and 4. During Night 5, the output pressure reaches the final therapy pressure PT, which is maintained during Night 6 and subsequent nights until the system is reset or changed. While only two sub-durations (e.g., D1 and D2) and one intermediate pressure (P2) are shown in FIG. 3, any number of sub-durations and intermediate pressures are contemplated by this embodiment.

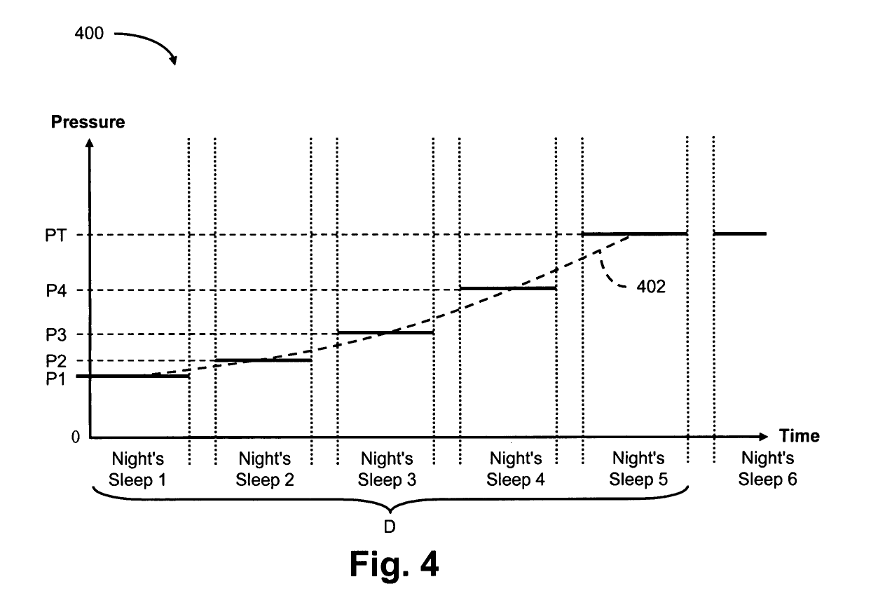

FIG. 4 illustrates an embodiment 400 where the change in pressure from initial pressure P1 to final therapy pressure PT follows a nonlinear profile 402. Nonlinear profile 402 may be, for example, exponential, logarithmic, parabolic, or any other nonlinear shape. In this embodiment, each pressure increment (P1, P2, P3, and P4) as a duration of one night's sleep though other durations are also contemplated as described above. During Night 1, the output pressure as at pressure P1. At Night 2, the output pressure is pressure P2. At Night 3, the output pressure is pressure P3. The change in pressure from initial pressure P1 to pressure P2 and from pressure P2 to pressure P3 is not constant. At Night 4, the output pressure is pressure P4. The change in pressure from pressure P3 to pressure P4 and from pressure P3 to pressure P2 is not constant as well. At Night 5, the output pressure is the final therapy pressure PT. The change in pressure from pressure P4 to the final therapy pressure PT and from pressure P4 to pressure P3 is also not constant. At Night 6 and for subsequent nights, the final therapy pressure PT is maintained until the system is reset or changed. Similar to FIG. 3, each pressure increment may have its own duration or sub-duration that is longer than one night's sleep.

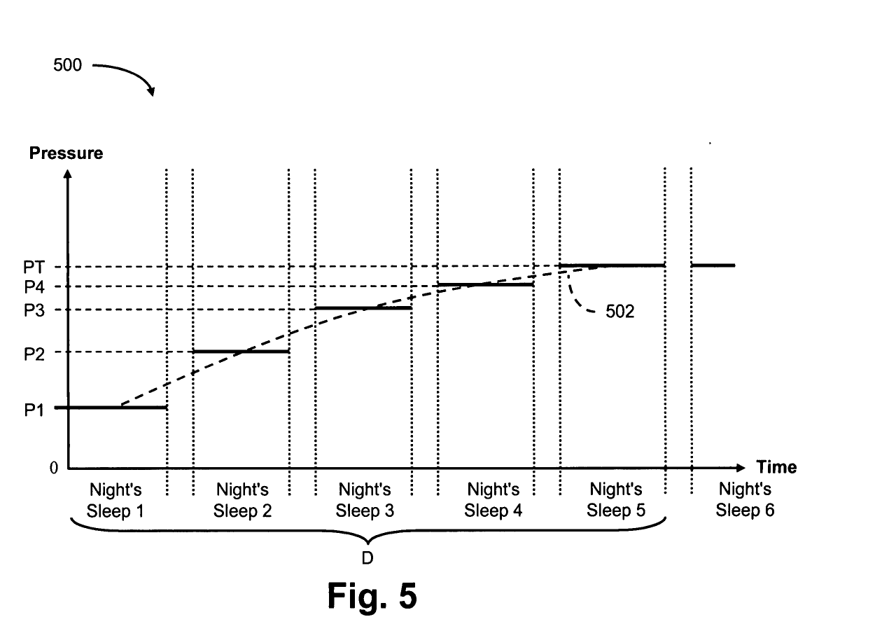

The embodiment of FIG. 4 illustrates changes in pressure that are initially small and grow larger as the final therapy pressure PT is approached. FIG. 5 illustrates an embodiment 500 where the changes in pressure are initially large and are reduced has the final therapy pressure PT is approached. Similar to FIG. 4, the differences in pressure between pressures P1 and P2, P2 and P3, P3 and P4, and P4 and PT are not constant but follow a nonlinear profile 502. Nonlinear profile 502 may be, for example, and inverted exponential, logarithmic, parabolic, or any other nonlinear shape.

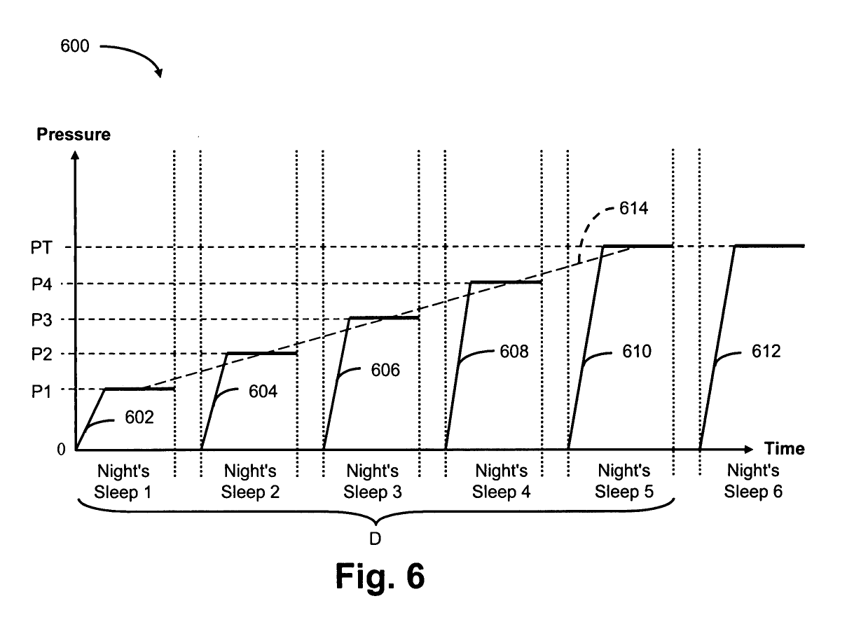

FIG. 6 illustrates an embodiment 600 similar to embodiment of 200, except that a ramp function has been added. The pressure for each night's sleep begins from an initial low pressure that may not necessarily be 0 or atmospheric and ramps up to the increment pressure. At Night 1, a ramp 602 is provided to gently raise the pressure to initial pressure P1. At Night 2, a ramp 604 is provided to gently raise the pressure to pressure P2. At Night 3, a ramp 606 is provided to gently raise the pressure to pressure P3. At Night 4, a ramp 608 is provided to gently raise the pressure to pressure P4. At Night 5, a ramp 610 is provided to gently raise the pressure to the final therapy pressure PT. At Night 6, ramp 612 raises the pressure in the same fashion as ramp 610 to the final therapy pressure PT. Ramps 602 through 610 may have a start pressure that is equal to atmosphere pressure or some pressure there above. For example, the starting ramp pressure may be the previous night's pressure increment. For example, ramp 604 may instead start from pressure P1 and rise to pressure P2. Alternatively, ramp 64 may have a starting pressure that is somewhere between atmospheric pressure (0) and pressure P1. Even though a ramp function has been added to the start of each night's sleep pressure increment, the pressure increments can still follow a linear pressure profile as shown by pressure profile 614.

Even though a ramp function has been added to each night's sleep pressure, the duration of the ramp function is a relatively short period of time compared to the duration of the night's sleep. Hence, even though a ramp function appears in the pressure output, the pressure is still considered to have been maintained for the night's sleep. Other variations in the magnitude of the pressure for purposes of comfort or compliance may be added to each night's sleep and fall within the meaning of maintaining a general pressure level or a mean pressure level during the night's sleep.

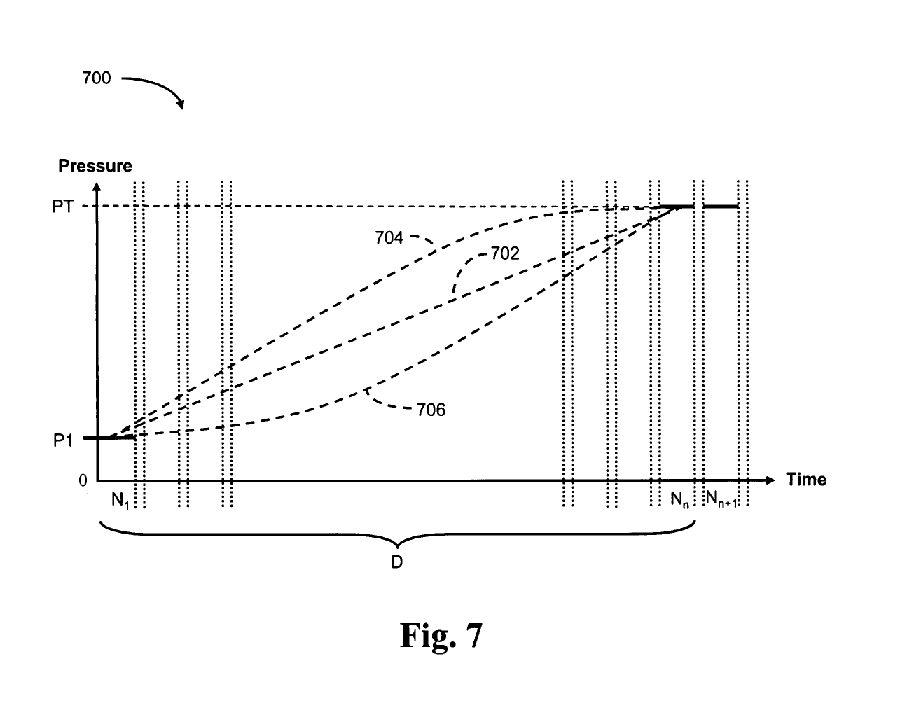

FIG. 7 illustrates a more general embodiment 700 of gas delivery. Embodiment 700 illustrates a plurality of possible pressure profiles that change the pressure from initial pressure P1 to final therapy pressure PT over a duration D of multiple nights (N₁ to Nₙ, where n can be any number). Pressure profile 702 is shown as a linear profile having a constant rate of change. With pressure profile 702, the pressure changes incrementally from initial pressure P1 to final therapy pressure PT via a plurality of constant pressure increments. The pressure change from night to night is the same until the final therapy pressure PT is reached.

Pressure profile 704 is shown as a nonlinear profile. Pressure profile 704 has a non-constant rate of change from initial pressure P1 to final therapy pressure PT. The changes in pressure from night to night are initially large starting from the initial night (N₁) and progressively diminish until the final therapy pressure PT is reached on the last night (Nₙ).

Pressure profile 706 is also shown as a nonlinear profile. Pressure profile 706 also has a non-constant rate of change from initial pressure P1 to final therapy pressure PT. The changes in pressure from night to night are initially small starting from the initial night (N₁) and progressively grow larger until the final therapy pressure PT is reached on last night (Nₙ).

Though pressure profiles 704 and 706 have been described as having a non-constant rate of change, it is contemplated that these pressure profiles may have portions thereof that include a constant rate of change and portions thereof that include a non-constant rate of change. For example, pressure profile 704 may have a portion thereof that includes a constant rate of change beginning from initial starting pressure P1 on night N₁ that changes to a portion thereof having in non-constant rate of change some nights later. Similarly, pressure profile 706 may have a portion thereof that includes a non-constant rate of change beginning from initial starting pressure P1 on night N₁ that changes to a portion thereof having in constant rate of change some nights later. Still further, pressure profiles 704 and 706 may include additional portions thereof having either constant or non-constant rates of change.

Referring now to FIG. 2C, an embodiment 204 illustrates pressure changes that are based on usage or compliance data thresholds. The rise from initial pressure P1 to final therapy pressure PT occurs over a duration D that is measured by usage or compliance data thresholds. The pressure output begins at pressure P1. Pressure P1 is maintained until a first usage or compliance threshold (e.g., “Usage Threshold 1”) is reached or exceeded. Thereafter, the pressure is increased to P2. Pressure P2 is maintained until a second usage or compliance threshold (e.g., “Usage Threshold 2”) is reached or exceeded. Thereafter, the pressure is increased to P3. Pressure P3 is maintained until a third usage or compliance threshold (e.g., “Usage Threshold 3”) is reached or exceeded, and so on until the final therapy pressure PT is attained. Once the final therapy pressure PT is attained, the output pressure remains at pressure PT until the system is reset or changed. The increases in pressure in this embodiment are represented by pressure profile 206, but may also take the form of pressure profiles 402, 502, and/or 614 (including the use of ramp features) of FIGS. 4, 5 and 6 respectively. The increases in pressure in this embodiment may also take the form of the pressure profile shown in FIG. 3.

As shown in FIG. 2C, a usage or compliance threshold may be shorter than one night's sleep. In such a scenario, the pressure output would increase from a first pressure (e.g., P1) to a second pressure (e.g., P2) during one night's sleep if the usage or compliance threshold has been exceeded during that one night's sleep. Still further, a timer may also be setup so that if the required usage or compliance threshold is not reached during a specific time period, the pressure output may be reduced. For example, if “Usage Threshold 2” is not reached in X number of hours, the output pressure is reduced from P2 to P1. The pressure would then remain at P1 until “Usage Threshold 1” is again exceeded so as raise the pressure back to pressure P2. Hence, the utilization of usage or compliance data can be employed to either raise or lower the pressure output.

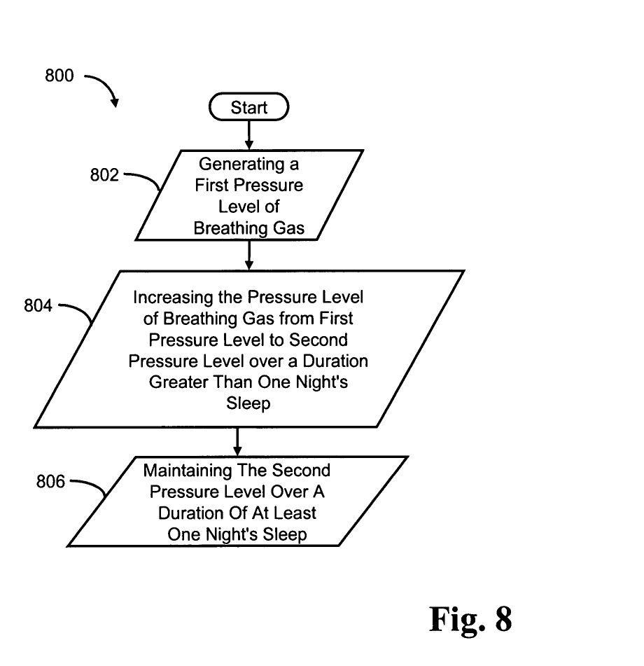

FIGS. 8 through 10 illustrate embodiments of flow diagrams 800, 900, and 1000 that can be implemented as control logic. The rectangular elements denote “processing blocks” and represent computer software instructions or groups of instructions. The diamond shaped elements denote “decision blocks” and represent computer software instructions or groups of instructions which affect the execution of the computer software instructions represented by the processing blocks. Alternatively, the processing and decision blocks represent steps performed by functionally equivalent circuits such as a digital signal processor circuit or an application-specific integrated circuit (ASIC). The flow diagram does not depict syntax of any particular programming language. Rather, the flow diagram illustrates the functional information one skilled in the art may use to fabricate circuits or to generate computer software to perform the processing of the system. It should be noted that many routine program elements, such as initialization of loops and variables and the use of temporary variables are not shown.

Flow diagram 800 illustrates one embodiment of control logic 104. In block 802, the system generates a first or initial pressure level of breathing gas (e.g., P1). In block 804, the pressure level of breathing gas is increased from the first or initial pressure level to a second pressure level over a duration equal to or greater than one night's sleep. In block 806, the second pressure level is maintained over a duration of least one night's sleep.

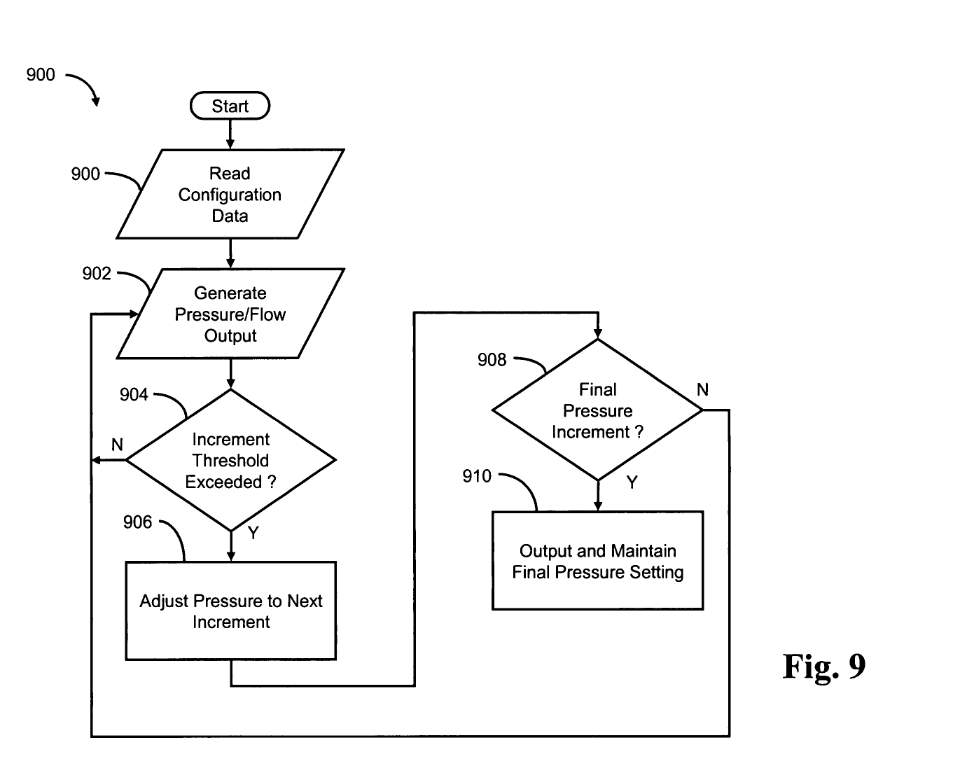

Referring now to FIG. 9, flow diagram 900 illustrates another embodiment of control logic 104. In block 900, configuration data is read from a memory or other device. The configuration data can include, for example, one or more of the following: initial or starting pressure, intermediate pressure, final or therapy pressure, duration, sub-durations, pressure change increment, increment threshold, pressure profile, ramp function profile, or ramp function modality (e.g., on or off). Other configuration data may also be added to the system.

In block 902, the system generates a pressure/flow output. For example, during the initial night of operation, the pressure output generated would generally be the starting or initial pressure P1. Block 904 tests to determine whether an increment threshold has been exceeded. The increment threshold determines whether conditions have been satisfied to increase the pressure from the present level to the next level or increment. The increment threshold may have several embodiments. In one embodiment, the increment threshold may take the form of a time duration such as, for example, one night's sleep. In another embodiment, the increment threshold may take the form of a level of compliance such as, for example, six hours of continuous therapy usage. In another embodiment, the increment threshold may be based on one or more physiological sensors connected to a patient. For example, the increment threshold may take the form of an apnea level that detects whether an apnea has occurred over a certain time duration. If the increment threshold is not exceeded in block 904, then the logic loops back to block 902 where the present pressure level is maintained.

If the increment threshold is exceeded in block 904, the logic of advances to block 906. In block 906, the pressure level is adjusted to the next increment because the increment threshold has been exceeded in block 904. The next pressure increment or pressure change may be defined by, for example, the type of pressure profile specified or other parameters including a specified pressure increment. In block 908, the logic determines whether the next pressure increment is the final pressure increment or final pressure. If the next pressure increment is not the final pressure, the logic loops back to block 902 where a pressure output is generated according to the next pressure increment. If the next pressure increment is the final pressure, the logic of advances to block 910 where the final pressure is output and maintained.

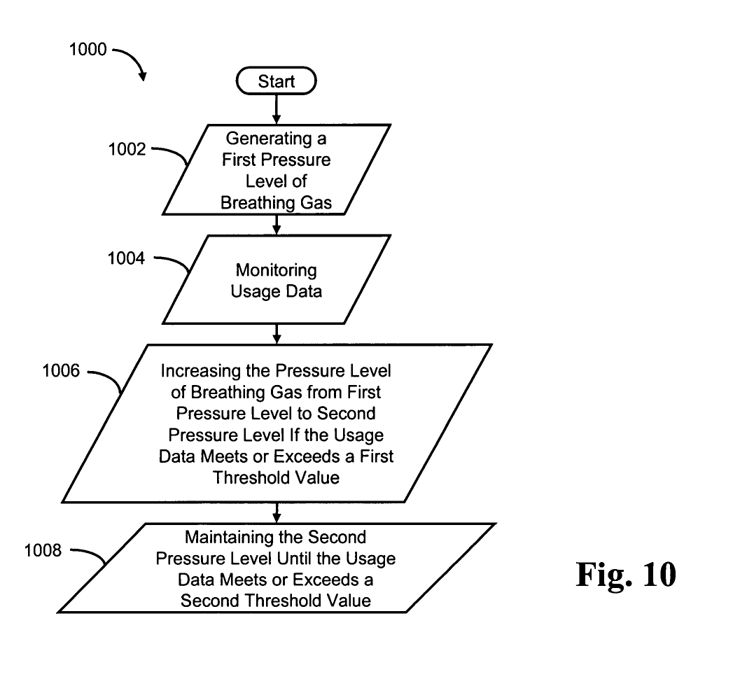

Illustrated in FIG. 10 is a flow diagram 1000 of another embodiment of control logic 104. In block 1002, the system generates a first or initial pressure level of breathing gas (e.g., P1). In block 1004, the usage or compliance data is monitored. Monitoring the usage or compliance data includes reading or obtaining one or more data values having usage or compliance data. In block 1006, the pressure level of breathing gas is increased from the first or initial pressure level (e.g., P1) to a second pressure level (e.g., P2) if the usage or compliance data meets or exceeds a first threshold value (e.g., Usage Threshold). In block 1008, the second pressure level is maintained until the usage or compliance data meets or exceeds a second threshold value. The first and second threshold values may have the same usage data value or different values. For example, the first and second thresholds may each have a value of 8 hours or the first threshold may have a value of 4 hours and the second threshold may have a value of 8 hours, or vice-versa.

The logic flow shown and described herein may reside in or on a computer readable medium or product such as, for example, a Read-Only Memory (ROM), Random-Access Memory (RAM), programmable read-only memory (PROM), electrically programmable read-only memory (EPROM), electrically erasable programmable read-only memory (EEPROM), magnetic disk or tape, and optically readable mediums including CD-ROM and DVD-ROM. Still further, the processes and logic described herein can be merged into one large process flow or divided into many sub-process flows. The process flows described herein may be rearranged, consolidated, and/or re-organized in their implementation as warranted or desired so long as the relative order is maintained. For example, other related or unrelated process flows can be interjected between the specified process blocks without affecting the functionality or results obtained.

While the present invention has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. For example, embodiments of the invention can be further modified to incorporate additional features such as proportional airway pressure, exhalation unloading, proportional assist ventilation, etc. Still further, the present invention may be applied to a bi-level pressure modality where each output pressure increment would have an inspiratory positive airway pressure (IPAP) and an expiratory positive airway pressure (EPAP). For example, in the Figures, initial pressure P1 may have an inspiratory pressure level (e.g., P1_{I}) and an expiratory pressure level (e.g., P1_{E}) where each inspiratory and expiratory pressure level is adjusted according the same approach as P1 would have been (e.g., by pressure increments). Therefore, the invention, in its broader aspects, is not limited to the specific details, the representative apparatus, and illustrative examples shown and described. Accordingly, departures may be made from such details without departing from the spirit or scope of the applicant's general inventive concept.

What is claimed:

1. A method of delivering a breathing gas comprising:
generating a first pressure level of breathing gas;
increasing the pressure level of the breathing gas from the first pressure level to a second pressure level over a duration greater than one night's sleep; and
maintaining the second pressure level over a duration of at least one night's sleep.

2. The method of claim 1 wherein the step of increasing the pressure level from the first pressure level comprises determining whether the first pressure level has a duration equal to or greater than a first time period.

3. The method of claim 1 wherein the step of increasing the pressure level from the first pressure level comprises incrementally increasing the pressure level.

4. The method of claim 1 wherein the step of increasing the pressure level from the first pressure level comprises increasing the pressure level to an intermediate pressure level that is less than the second pressure level.

5. The method of claim 1 wherein the step of increasing the pressure level from the first pressure level comprises incrementally increasing the pressure level to an intermediate pressure level that is less than the second pressure level.

6. The method of claim 1 wherein the step of increasing the pressure level from the first pressure level comprises increasing the pressure level to at least one of a plurality of intermediate pressure levels that are less than the second pressure level.

7. The method of claim 1 wherein the step of increasing the pressure level from the first pressure level comprises incrementally increasing the pressure level to at least one of a plurality of intermediate pressure levels that are less than the second pressure level.

8. The method of claim 1 wherein the step of increasing the pressure level from the first pressure level comprises increasing the pressure level according to a ramp function.

9. The method of claim 1 wherein the step of increasing the pressure level from the first pressure level comprises linearly increasing the pressure level.

10. The method of claim 1 wherein the step of increasing the pressure level of the breathing gas from the first pressure level to a second pressure level over a duration equal to or greater than one night's sleep comprises increasing the pressure level over a duration of a plurality of nights' sleep.

11. The method of claim 1 wherein the step of increasing the pressure level of the breathing gas from the first pressure level to a second pressure level comprises monitoring usage data.

12. The method of claim 1 wherein the step of increasing the pressure level of the breathing gas from the first pressure level to a second pressure level comprises monitoring usage data and increasing the pressure level of the breathing gas if the usage data exceeds or fails to exceed or reach a threshold value.

13. The method of claim 1 wherein the step of increasing the pressure level of the breathing gas from the first pressure level to a second pressure level comprises monitoring sensor data and increasing the pressure level of the breathing gas if the sensor data exceeds or fails to exceed or reach a threshold value.

14. The method of claim 1 wherein the step of increasing the pressure level from the first pressure level to a second pressure level comprises increasing the pressure level in non-linear pressure increments.

15. The method of claim 1 wherein the step of increasing the pressure level from the first pressure level to a second pressure level comprises increasing the pressure level in logarithmic pressure increments.

16. The method of claim 1 wherein the step of increasing the pressure level from the first pressure level to a second pressure level comprises increasing the pressure level in exponential pressure increments.

17. A system for delivering a breathing gas comprising:
a flow generator;
a controller having:
a first set of instructions that generate a first pressure level of breathing gas;
a second set of instructions that increase the pressure level of the breathing gas from the first pressure level to a second pressure level over a duration greater than one night's sleep; and
a third set of instructions that maintain the second pressure level over a duration of at least one night's sleep.

18. The system of claim 17 wherein the second set of instructions that increase the pressure level of the breathing gas from the first pressure level to a second pressure level over a duration greater than one night's sleep comprises instructions that increase the pressure level over a duration of a plurality of nights' sleep.

19. A system for delivering a breathing gas comprising:
means generating a first pressure level of breathing gas;
means increasing the pressure level of the breathing gas from the first pressure level to a second pressure level over a duration greater than one night's sleep; and
means maintaining the second pressure level over a duration of at least one night's sleep.

20. The system of claim 19 wherein the means for increasing the pressure level of the breathing gas from the first pressure level to a second pressure level over a duration equal to or greater than one night's sleep comprises means increasing the pressure level over a duration of a plurality of nights' sleep.

21. A method of delivering a breathing gas comprising:
generating a first pressure level of breathing gas;
monitoring usage data;
increasing the pressure level of the breathing gas from the first pressure level to a second pressure level if the usage data meets or exceeds a first threshold value; and
maintaining the second pressure level until the usage data meets or exceeds a second threshold value.

22. The method of claim 21 wherein the first and second thresholds have the same usage data value.

23. The method of claim 21 wherein the first and second thresholds have different usage data values.

24. The method of claim 21 further comprising the step of changing the second pressure level if the usage data meets or exceeds the second threshold value.

25. The method of claim 21 further comprising the step of reducing the pressure below the second pressure level if the usage data does not meet or exceed the second threshold over a time period.

26. The method of claim 21 further comprising the step of increasing the pressure above the second pressure level if the usage data meets or exceeds the second threshold value.
